(19) United States
(12) Patent Application Publication
(10) Pub. No.: US 2022/0266047 A1
(43) Pub. Date: Aug. 25, 2022

(54) SKIN TREATMENT DEVICE FOR THERMOTHERAPY

(71) Applicant: K CO., LTD., Hwaseong-si (KR)

(72) Inventors: Jung Rack KIM, Hwaseong-si (KR); Dong Hoo KIM, Osan-si (KR); Ji An KIM, Gwacheon-si (KR)

(73) Assignee: K CO., LTD., Hwaseong-si (KR)

(21) Appl. No.: 17/674,883

(22) Filed: Feb. 18, 2022

(30) Foreign Application Priority Data
Feb. 19, 2021 (KR) 10-2021-0022568

(51) Int. Cl. A61N 1/40

(52) U.S. Cl. CPC A61N 1/403

(57) ABSTRACT

The present invention is to solve the problems of the prior arts as described above and to achieve a more effective skin treatment effect by thermal therapy, wherein radio-frequency electrical energy is transferred to electrodes in contact with the skin and heat is applied to tissue under the skin by electric fields formed by the electrodes. The skin treatment device for thermotherapy according to the present invention has advantages in that it is possible to provide various operation modes such as an operation mode for skin beauty effects and an operation mode for reducing body fat by controlling a depth for generating heat under the skin by changing the positions of the bipolar electrode-pairs of the electrodes in contact with the skin,

### BACKGROUND OF THE INVENTION

### Field of the Invention

The present invention relates to a skin treatment device for thermotherapy, and more specifically, the skin treatment device for thermotherapy according to the present invention relates to an apparatus capable of preventing skin aging and improving skin elasticity by applying deep heat by radio-frequency energy to tissues in the skin of the face, body, etc.

### Description of the Related Art

In general, as a person's skin ages, elasticity decreases and the skin stretches and wrinkles, and when exposed to UV light for a long time, the skin becomes thicker and the skin's blood circulation ability decreases, and moisture and nutrients are not supplied smoothly to the skin.

In order to solve this problem, a functional substance such as a nourishing cream has been applied to a skin, or a skin massage method for providing a physical stimulation to the skin has been used to further facilitate blood circulation.

In addition, it is already known that the radio-frequency electric stimulation of the skin promotes absorption of nutrients applied to the skin and promotes blood circulation by generating deep heat in the skin.

A number of prior art documents related to a skin beauty device to simultaneously provide massage and radio-frequency electric stimulation for the skin have been published, Korean patent publications No. 10-2020-0101170, No. 10-2017-0068697, No. 10-2016-0086275.

Prior arts as described above are provided with a plurality of electrodes on the surface in contact with the skin to provide high-frequency electricity to the contact surface of the skin through the electrodes to promote absorption of creams and drugs applied to the skin, and deep heat to the skin can promote blood circulation.

It is known that by generating deep heat in the dermal layer under the epidermis of the skin, the temperature of tissue in the dermal layer can be increased, and skin beauty effects can be obtained by improving cell function and increasing blood flow, and by generating deep heat in the lower dermal layer or underneath the skin, blood vessels can be expanded to increase the amount of oxygen and burn fat to reduce body fat.

That is, according to the depth of generating deep heat under the skin's epidermis, the skin beauty effect as described above may be obtained or the body fat reduction effect as described above may be obtained.

Conventionally there were radio-frequency heating devices as a skin beauty device that generates deep heat in the dermal layer under the skin's epidermis and as a device for reducing body fat that generates deep heat on or below the dermal layer of the skin, separately.

Thus, as a consumer, in order to obtain the effect of skin beauty and body fat reduction, there was a problem that consumers had to purchase and use a device for skin beauty and a device for body fat reduction, separately.

### SUMMARY OF THE INVENTION

The present invention is to solve the problems of the prior arts as described above and to achieve a more effective skin treatment effect by thermal therapy, wherein radio-frequency electrical energy is transferred to electrodes in contact with the skin and heat is applied to tissue under the skin by electric fields formed by the electrodes.

In accordance with one aspect of the present invention, the above and other objects can be accomplished by the provision of a skin treatment device for thermotherapy comprising: a handpiece: a head provided on a distal end of the handpiece, wherein the head has an electrode contact including a plurality of electrodes in contact with a skin and transferring heat to tissues in the skin according to radio-frequency electric signal applied to each of the plurality of the electrodes; a signal operator that generates and connects the radio-frequency electric signal to each of the electrodes of the electrode contact; and a controller that controls the signal operator to make one of the electrodes into a common electrode and each of the other electrodes into an operating electrode so that the common electrode and each of the operating electrodes form a bipolar electrode-pair and the bipolar electrode-pair transfers heat by the radio-frequency electric signal to tissues in the skin, wherein the controller controls the signal operator to change a position of the common electrode.

Preferably, the controller is configured to control the signal operator to adjust a depth at which heat is transferred by the radio-frequency in tissues in the skin by changing an electrode to which a signal for the common electrode is transmitted.

Preferably, the signal operator comprises: a signal generator that generates an electric signal for the common electrode and an electric signal for each of the operating electrodes; and a signal connector that connects an electrical signal for the common electrode to an electrode specified by the controller among the plurality of the electrodes of the electrode contact and connects electrical signals for each of the plurality of operating electrodes to the other electrodes other than the specified electrode, respectively, so that the common electrode and each of the operating electrodes form a bipolar electrode-pair.

Preferably, the controller is configured to provide a plurality of operation modes according to a depth at which heat is transferred by the radio-frequency in tissues in the skin, and control the signal operator to allow an electric signal for the common electrode to be connected to an electrode selected from the plurality of the electrodes of the electrode contact according to an operation mode selected from the plurality of the operation modes.

Preferably, the controller is configured to control the signal operator to change the position of the common electrode so that the distance between the common electrode and at least one of the operating electrodes forming the bipolar electrode-pair is farther as the depth of heat transferred to the tissue in the skin is deeper.

Preferably, the controller is configured to control the depth of heat generated by the electrode contact in the tissue in the skin by adjusting the distance between the common electrode and each of the operating electrodes forming the bipolar electrode-pair.

Preferably, the signal operator includes a plurality of channels so that each of the plurality of the channels is connected to each of the plurality of electrodes of the electrode contact.

Preferably, the signal operator is configured to transmit the radio-frequency electrical signal to each electrode through each channel according to the control of the controller.

Preferably, the signal operator is configured to transmit an electric signal for the common electrode through a channel selected to make an electrode selected by the controller into the common electrode.

Preferably, the signal operator is configured to allow one of the plurality of electrodes of the electrode contact to be the common electrode through one of the channels, and allow each of the other electrodes of the electrode contact to be the operating electrode through each of the other channels, so that the common electrode and each of the operating electrodes form a bipolar electrode-pair.

Preferably, the signal operator is configured to apply AC current the common electrode and each operating electrode as bipolar electrode-pairs so that a direction of electric fields of the bipolar electrode-pairs are consecutively changed and generate deep heat in the tissue in the skin.

Preferably, the electrode contact of the head includes a plurality of circular electrodes as the plurality of the electrodes and is configured such that the plurality of circular electrodes form a concentric circle at predetermined intervals, wherein the controller is configured to control the signal operator to make a circular electrode selected from the plurality of the circular electrodes into the common electrode and make each of the other circular electrodes into the operating electrode so that the common electrode and each of the operating electrodes form a bipolar electrode-pair, and wherein the controller is configured to control the depth at which heat generated by the bipolar electrode-pair is transferred in the tissue in the skin by adjusting a distance between the common electrode and the operating electrode

The skin treatment device for thermotherapy according to the present invention has advantages in that it is possible to provide various operation modes such as an operation mode for skin beauty effects and an operation mode for reducing body fat by controlling a depth for generating heat under the skin by changing the positions of the bipolar electrode-pairs of the electrodes in contact with the skin,

### BRIEF DESCRIPTION OF THE DRAWINGS

FIG. 1 shows perspective views (a) and (b) of the skin treatment device according to an embodiment of the present invention viewed from different perspectives.

FIG. 2 shows the electrode contact of the head of the skin treatment device shown in FIG. 1 and configurations related to the electrode contact of the head.

FIG. 3 shows (a) and (b) illustrating a configuration, an operation and an effect according to a first operation mode described above with respect to the skin treatment device for thermotherapy according to an embodiment of the present invention.

FIG. 4 shows (a) and (b) illustrating a configuration, an operation and an effect according to a second operation mode described above for the skin treatment device for the thermotherapy according to an embodiment of the present invention.

### DESCRIPTION OF THE PREFERRED EMBODIMENTS

Hereinafter, specific details of the skin treatment device for thermotherapy according to the present invention will be described with reference to the drawings.

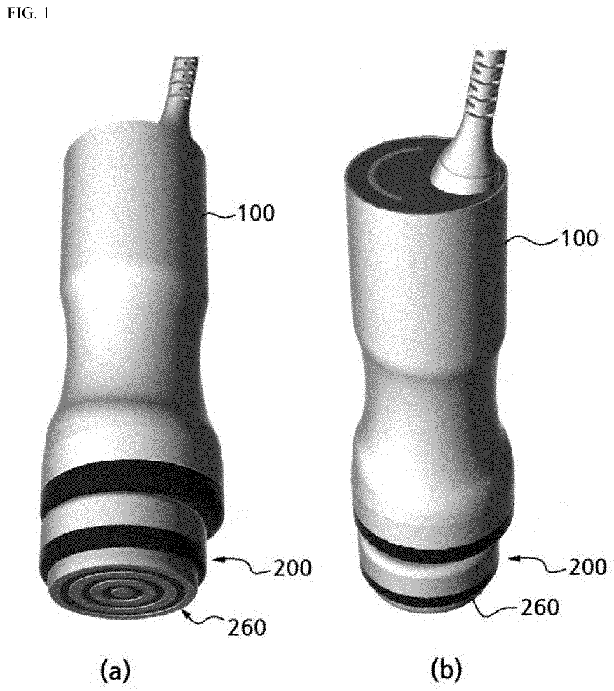

First, an overall configuration of the skin treatment device for thermotherapy according to an embodiment of the present invention will be described with reference to FIG. 1. FIG. 1(a) and (b) respectively show a perspective view of the skin treatment device according to an embodiment of the present invention viewed from different perspectives.

As shown in FIG. 1(a) and (b), the skin treatment device for thermotherapy according to an embodiment of the present invention is provided with a handpiece 100 provided so that a user can grip.

The handpiece 100 has a proximal end and a distal end as shown in FIG. 1, and a grip portion concavely formed so that a user can easily grip it.

A head 200 for generating deep heat due to radio frequency electrical signal by contacting the skin may be provided on the distal end of the handpiece 100.

An electrode contact 300 including a plurality of electrodes contacting the skin may be provided at an end of the head 200.

While the user holds the handpiece 100 and contacts the electrode contact 300 of the head 200 with the skin, the electrode contact 300 may generate deep heat in the skin by a radio frequency electrical signal transmitted from a signal operator (not shown).

The signal operator (not shown) is a device that generates a radio-frequency electric signal so that the electrode contact contacts the skin and applies radio-frequency heat to the tissue in the skin. The signal operator (not shown) may be provided outside the handpiece 100, and may be electrically connected to the handpiece 100 by a cable.

The handpiece 100 may be configured to be electrically connected to a main device (not shown). The signal operator (not shown) may be provided in the main device. In addition to the signal operator, a power supply device may be provided in the main device to connect power from the main device to the handpiece through a cable connected to the handpiece 100. A controller (not shown) can be provided in the main device, the controller can control the signal operator from generating a radio-frequency electrical signal and control the connection between the radio-frequency electrical signal and each of the electrodes of the electrode contact.

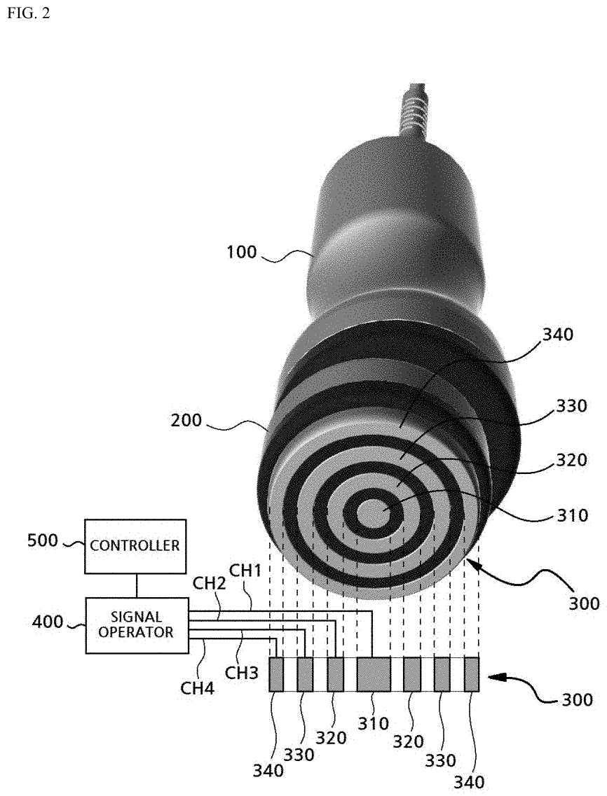

Meanwhile, the configuration and control system of the skin treatment device for thermotherapy according to an embodiment of the present invention will be described with reference to FIG. 2. FIG. 2 shows the electrode contact of the head of the skin treatment device shown in FIG. 1 and configurations related to the electrode contact of the head.

As shown in FIG. 2, the electrode contact 300 of the head 200 of the skin treatment device for thermotherapy according to an embodiment of the present invention includes a plurality of electrodes 310, 320, 330 and 340 may be in contact with the skin, and may transfer radio-frequency heat to the tissue in the skin according to radio-frequency electrical signals applied to each of the electrodes 310, 320, 330, and 340.

As shown in FIG. 2, the electrode contact 300 of the head 200 may be configured such that a plurality of circular electrodes 310, 320, 330, and 340 form concentric circles at predetermined intervals. FIG. 2 shows a cross-section of the electrode contact 300 of the head 200, wherein a plurality of electrodes 310, 320, 330, and 340 may be arranged in a row at predetermined intervals.

Each of a plurality of electrodes 310, 320, 330 and 340 of the electrode contact 300 may be electrically connected to a signal operator 400, and the signal operator 400 generates a bipolar radio-frequency electric signal to be connected to each of the electrodes 310, 320, 330 and 340.

For example, as shown in FIG. 2, the electrode contact 300 may be configured such that a first electrode 310, a second electrode 320, a third electrode 330, and a fourth electrode 340 are provided at predetermined intervals to simultaneously contact the skin. And, the signal operator 400 may include a first channel CH1 electrically connected to the first electrode 310, a second channel CH2 electrically connected to the second electrode 320, a third channel CH3 electrically connected to the third electrode 330, and a fourth channel CH4 electrically connected to the fourth electrode 340.

Each of the first to fourth channels CH1 to CH4 of the signal operator 400 are electrically connected to each of the plurality of electrodes 310 to 340 of the electrode contact 300, and the electrical connection thereof may be connected by various means.

For example, each of the channels CH1 to CH4 of the signal operator 400 may be a wire connected to each of the electrodes, an electrical contact connection means such as a Pogo pin, or a contact terminal in electrical contact with each of the electrodes.

The signal operator 400 may allow one of the plurality of electrodes of the electrode contact 300 to be a ‘common electrode’ through one of the channels, and allow each of the other electrodes of the electrode contact 300 to be an ‘operating electrode’ through each of the other channels, so that the common electrode and each of the operating electrodes form a bipolar electrode-pair.

For example, the controller 500 may control the signal operator 400 to transfer an electric signal for a common electrode through the fourth channel CH4 so that the fourth electrode 340 connected to the fourth channel CH4 becomes the common electrode, transfer an electric signal for a first operating electrode through the first channel CH1 of the signal operator 400 so that the first electrode 310 connected to the first channel CH1 becomes the first operating electrode, transfer an electric signal for a second operating electrode through the second channel CH2 of the signal operator 400 so that the second electrode 320 connected to the second channel CH2 becomes the second operating electrode, and transfer an electrical signal for a third operating electrode through the third channel CH3 of the signal operator 400 so that the third electrode 330 connected to the third channel CH3 becomes the third operating electrode.

The common electrode 340 and the first operating electrode 310 may form a bipolar electrode-pair, the common electrode 340 and the second operating electrode 320 may form another bipolar electrode-pair, and the common electrode 340 and the third operating electrode 330 may form the other bipolar electrode-pair.

As described above, the common electrode and each operating electrode form bipolar electrode-pairs, and an electric field is formed between the common electrode and each operating electrode according to the application of AC current. And the direction of the electric field changes according to the frequency of the AC current, thereby generating deep heat in the tissue in the skin.

In this case, the depth of heat in the tissue in the skin can be changed according to the distances between the common electrode and each operating electrode.

The closer the distance between the common electrode and the operating electrode is, the shallower the heat is generated in the tissue in the skin, and the farther the distance between the common electrode and the operating electrode is, the deeper the heat is generated in the tissue in the skin.

The skin treatment device for thermotherapy in accordance with an embodiment of the present invention may provide a plurality of operation modes, and may vary the depth of the thermal effects generated by the electrode contact 300 to tissues in the skin according to the operation modes.

The controller 500 controls the signal operator 400 to allow at least one of the plurality of electrodes 310, 320, 330, and 340 of the electrode contact 300 to become the common electrode, and each of the other electrodes to be an operating electrode, and each of the operating electrodes makes a bipolar electrode-pair with the common electrode, respectively, so that heat by radio-frequency may be transferred to tissue in the skin through each of the bipolar electrode-pairs.

The skin treatment device for thermotherapy according to an embodiment of the present invention provides a plurality of operation modes according to a depth at which heat by radio-frequency is transferred in the skin. The controller 500 controls the position of the common electrode to be changed according to the operation mode, so that the electrode contact 300 may adjust a depth at which heat by radio-frequency is transferred in the skin according to the operation mode.

In the case of the operation mode in which heat is transferred to the deep inside of the skin among the plurality of operation modes (e.g., in the case of an operation mode for reducing body fat, hereinafter referred to as a “first operation mode”), the controller 500 may determine a position to be the common electrode among the plurality of electrodes so that the distance between the common electrode and one of the operating electrodes is the longest (when the position of the common electrode is determined, the remaining electrodes are determined as operating electrodes).

In the case of the operation mode in which heat is transferred to the shallow inside of the skin among the plurality of operation modes (e.g., in the case of an operation mode for skin beauty effects, hereinafter referred to as a “second operation mode”), the controller 500 may determine a position to be the common electrode among the plurality of electrodes so that the longest distance among the distances between the common electrode and each of the operating electrodes is shorter than the longest distance between the common electrode and the operating electrode in the first operating mode (when the position of the common electrode is determined, the remaining electrodes are determined as operating electrodes).

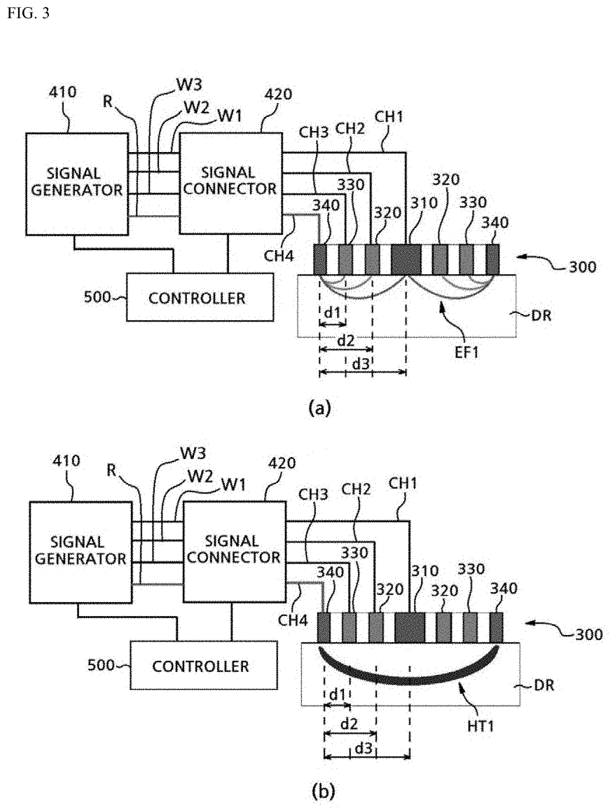

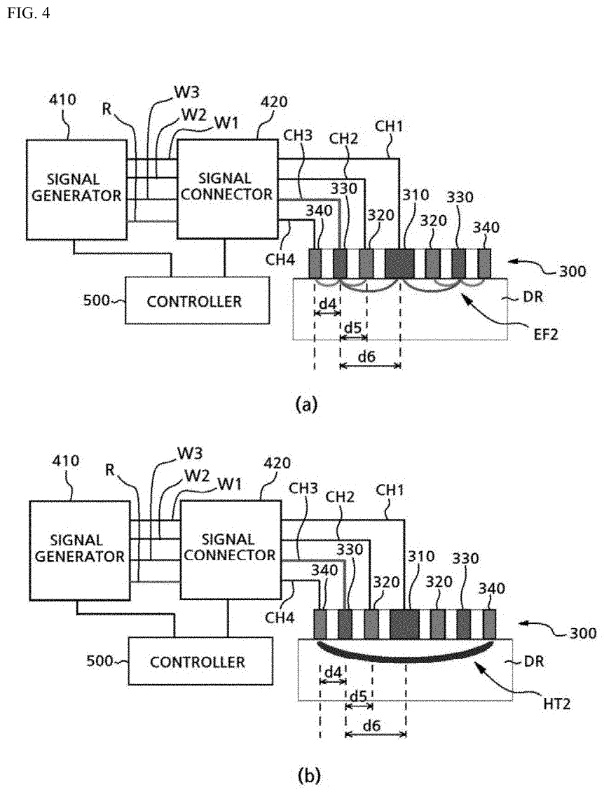

In this regard, the operation modes of the skin treatment device for thermotherapy according to an embodiment of the present invention will be described specifically with reference to FIGS. 3 and 4.

FIG. 3 shows a configuration, an operation and an effect according to the first operation mode described above with respect to the skin treatment device for thermotherapy according to an embodiment of the present invention. FIG. 3(a) shows a view in which electric fields are formed between the plurality of the electrodes in the first operation mode, and FIG. 3(b) shows a view in which heat is generated deep in the skin in the state shown in FIG. 3(a).

In addition, FIG. 4 shows a configuration, an operation and an effect according to the second operation mode described above for the skin treatment device for the thermotherapy according to an embodiment of the present invention. FIG. 4(a) shows a view in which electric fields are formed between the plurality of the electrodes in the second operation mode, and FIG. 4(b) shows a view in which heat is generated shallow in the skin in the state shown in FIG. 4(a).

The skin treatment device for thermotherapy according to an embodiment of the present invention has a configuration where all of the electrode contact 300, the signal operator 400, and the controller 500 are connected, and the signal operator 400 may include a signal generator 410 and a signal connector 420 as shown in FIGS. 3 and 4.

The signal generator 410 is configured to generate an electrical signal for the common electrode and electrical signals for each of the plurality of operating electrodes. The signal connector 420 connects an electrical signal for the common electrode to an electrode specified by the controller 500 among the plurality of electrodes of the electrode contact 300 under the control of the controller 500.

In addition, the signal connector 420 connects electrical signals for each of the plurality of operating electrodes to the remaining electrodes other than the specified electrode, respectively, so that the common electrode and each of the operating electrodes form a bipolar electrode-pair.

As shown in FIG. 3, the signal generator 410 may include a number of signal lines R, W1, W2, and W3 corresponding to the number of electrodes 310, 320, 330, and 340 of the electrode contact 300. That is, the signal line R for the common electrode and the signal lines W1, W2, and W3 for the operating electrodes may be provided. The signal lines may be provided with an electrical connection means such as wire, Pogo-pin, or the like.

The signal generator 410 may generate a radio-frequency electrical signal for the common electrode through the R signal line, generate a radio-frequency electrical signal for the first operating electrode through the W1 signal line, generate a radio-frequency electrical signal for the second operating electrode through the W2 signal line, and generate a radio-frequency electrical signal for the third operating electrode through the W3 signal line.

Radio-frequency electrical signals through each of the signal lines R, W1, W2, and W3 are connected to the signal connector 420. The signal connector 420 is electrically connected to each of the electrodes 310, 320, 330, and 340 of the electrode contact 300 by the plurality of channels CH1, CH2, CH3, and CH4, respectively. The signal connector 420 may connect an electrical signal for the common electrode to an electrode selected through one of the channels under the control of the controller 500, and connect electrical signals for the operating electrodes to the remaining electrodes through the remaining channels, respectively.

As shown in FIG. 3, electrical signals generated by the signal generator 410 may be transmitted to each electrode through channels of the signal connector 420. Wherein the signal connector 420 may selectively connect the electrical signal to each electrode by selecting a channel for transmitting the electrical signal from the signal generator 410 to each electrode.

For example, the signal connector 420 may connect a signal for the common electrode transmitted through an R signal line of the signal generator 410 to a channel selected from among the plurality of channels to transmit the same to the electrode contact, and may include a switch or relay selectively connecting the signals between the two terminals.

As shown in FIG. 3, the first channel CH1 of the signal connector 420 may be connected to the first electrode 310 of the electrode contact 300, the second channel CH2 of the signal connector 420 may be connected to the second electrode 320 of the electrode contact 300, the third channel CH3 of the signal connector 420 may be connected to the third electrode 330 of the electrode contact 300, and the fourth channel CH4 of the signal connector 420 may be connected to the fourth electrode 340 of the electrode contact 300.

The controller 500 may select one channel from among the first to fourth channels CH1 to CH4 according to the operation mode so that the common electrode signal transmitted through the R signal line of the signal generator 410 is connected to the corresponding electrode through the selected channel, and allow the plurality of operating electrode signals transmitted through W1 to W3 signal lines of the signal generator 410 to be connected to each corresponding electrode through the remaining channels.

For example, as shown in FIG. 3(a), the controller 500 may control the signal generator 410 to generate a radio-frequency electric signal for the common electrode through the R signal line and generate radio-frequency electric signals for the first to third operating electrodes through the W1 to W3 signal lines. The controller 500 may control the signal connector 420 to connect the signal of the common electrode through the R signal line to the fourth electrode 340 through the fourth channel CH4, to connect the signal of the first operating electrode through the W1 signal line to the first electrode 310 through the first channel CH1, to connect the signal of the second operating electrode through the W2 signal line to the second electrode 320 through the second channel CH2, and to connect the signal of the third operating electrode through the W3 signal line to the third electrode 330 through the third channel CH3.

Accordingly, in a state in which the electrode contact 300 contacts the skin SK, electric fields EF1 are formed between the common electrode (the fourth electrode 340) and the first operating electrode (the first electrode 310), between the common electrode (the fourth electrode 340) and the second operating electrode (the second electrode 320), and between the common electrode (the fourth electrode 340) and the third operating electrode (the third electrode 330), respectively.

The common electrode (the fourth electrode 340) and each of the operating electrodes (the first to third electrodes 310, 320 and 330) form different polarities to each other, and the polarity continues to change depending on the frequency of the AC current. Accordingly, as the direction of the electric fields EF1 are consecutively changed, heat HT1 may be applied to the tissue DR in the skin as shown in FIG. 3(b).

As described above, the skin treatment device for thermotherapy according to an embodiment of the present invention may provide a first operation mode with a deeper depth of heat transfer and a second operation mode with a shallower depth of heat transfer, according to a depth of heat transfer in tissues in the skin, FIGS. 3(a) and 3(b) show an example of the first operation mode.

The first operation mode providing a deeper depth of heat transfer in the skin and the second operation mode providing a shallower depth of heat transfer in the skin may be distinguished according to a distance between the common electrode and one of the operation electrodes.

As shown in FIGS. 3(a) and 3(b), in the first operation mode, the distance between the common electrode 340 and the third operating electrode 330 is d1, the distance between the common electrode 340 and the second operating electrode 320 is d2, and the distance between the common electrode 340 and the first operating electrode 310 is d3, wherein the distance d3 is the longest among the distances between the common electrode and each of the operating electrodes, and is longer than the longest distance between a common electrode and an operating electrode in the second operation mode.

The longer the distance between the common electrode and the operating electrode, the deeper the heat generated by the electric field between the two electrodes can be transferred to the tissue in the skin. Accordingly, since the maximum distance between the common electrode and the operating electrode in the first operation mode is longer than the maximum distance between the common electrode and the operating electrode in the second operation mode, in the first operation mode heat may be transferred to a deeper position of tissue in the skin (in the first operation mode, thermal effect by radio-frequency electrical signal can be applied to a deeper position in the skin).

Meanwhile, FIG. 4 shows the case of the second operation mode described above. The configuration of the electrode contact 300, the signal generator 410, the signal connector 420, and the controller 500 shown in FIG. 4 is substantially the same as that shown in FIG. 3, and thus a detailed description thereof will be omitted.

As shown in FIG. 4, the signal generator 410 may generate a radio-frequency electrical signal for the common electrode through the R signal line, generate a radio-frequency electrical signal for the first operating electrode through the W1 signal line, generate a radio-frequency electrical signal for the second operating electrode through the W2 signal line, and generate a radio-frequency electrical signal for the third operating electrode through the W3 signal line.

Radio-frequency electrical signals through each of the signal lines R, W1, W2, and W3 are connected to the signal connector 420. The signal connector 420 is electrically connected to each of the electrodes 310, 320, 330, and 340 of the electrode contact 300 by the plurality of channels CH1, CH2, CH3, and CH4, respectively. The signal connector 420 may connect an electrical signal for the common electrode to an electrode selected through one of the channels under the control of the controller 500, and connect electrical signals for the operating electrodes to the remaining electrodes through the remaining channels, respectively.

The controller 500 may select one channel from among the first to fourth channels CH1 to CH4 according to the operation mode so that the common electrode signal transmitted through the R signal line of the signal generator 410 is connected to the corresponding electrode through the selected channel, and allow the plurality of operating electrode signals transmitted through W1 to W3 signal lines of the signal generator 410 to be connected to each corresponding electrode through the remaining channels.

For example, as shown in FIG. 4(a), the controller 500 may control the signal generator 410 to generate a radio-frequency electric signal for the common electrode through the R signal line and generate radio-frequency electric signals for the first to third operating electrodes through the W1 to W3 signal lines. The controller 500 may control the signal connector 420 to connect the signal of the common electrode through the R signal line to the third electrode 330 through the third channel CH3, to connect the signal of the first operating electrode through the W1 signal line to the first electrode 310 through the first channel CH1, to connect the signal of the second operating electrode through the W2 signal line to the second electrode 320 through the second channel CH2, and to connect the signal of the fourth operating electrode through the W4 signal line to the fourth electrode 340 through the fourth channel CH4.

Accordingly, in a state in which the electrode contact 300 contacts the skin SK, electric fields EF2 are formed between the common electrode (the third electrode 330) and the first operating electrode (the first electrode 310), between the common electrode (the third electrode 330) and the second operating electrode (the second electrode 320), and between the common electrode (the third electrode 330) and the fourth operating electrode (the fourth electrode 340), respectively.

The common electrode (the third electrode 330) and each of the operating electrodes (the first, second, and fourth electrodes 310, 320 and 340) form different polarities to each other, and the polarity continues to change depending on the frequency of the AC current. Accordingly, as the direction of the electric fields EF2 are consecutively changed, heat HT2 may be applied to the tissue DR in the skin as shown in FIG. 4(b).

FIGS. 3(a) and 3(b) show an example of a case in the first operation mode, and FIGS. 4(a) and 4(b) show an example of a case in the second operation mode.

In the first operation mode, the depth at which heat is transferred to the tissue in the skin is deeper, and in the second operation mode, the depth at which heat is transferred to the tissue in the skin is shallower.

In the first operation mode, as shown in FIG. 3, the controller 500 controls the signal connector 420 to become the fourth electrode 340 to the common electrode through the fourth channel CH4, and the first electrode 310, the second electrode 320 and the third electrode 330 to the operating electrodes through the first channel CH1, the second channel CH2 and the third channel CH3, respectively, so that heat HT1 by the electric field EF1 generated between the common electrode (the fourth electrode 340) and each of the operating electrodes (the first, the second and the third electrodes 310, 320 and 330) can be applied in the skin, wherein the distances between the common electrode 340 and each of the operating electrodes 310, 320 and 330 are d1, d2 and d3, respectively.

While, in the second operation mode, as shown in FIG. 4, the controller 500 controls the signal connector 420 to become the third electrode 330 to the common electrode through the third channel CH3, and the first electrode 310, the second electrode 320 and the fourth electrode 340 to the operating electrodes through the first channel CH1, the second channel CH2 and the fourth channel CH4, respectively, so that heat HT2 by the electric field EF2 generated between the common electrode (the third electrode 330) and each of the operating electrodes (the first, the second and the fourth electrodes 310, 320 and 340) can be applied in the skin, wherein the distances between the common electrode 330 and each of the operating electrodes 310, 320 and 340 are d4, d5 and d6, respectively.

As described above, comparing the distances d1, d2, and d3 between the common electrode and each of the operating electrodes in the first operation mode and the distances d4, d5, and d6 between the common electrode and each of the operating electrodes in the second operation mode, the distance d1 is equal to the distance d4 and d5, and the distance d2 is equal to the distance d6, however the distance d3 is longer than the distance d6.

Since the depth at which heat is transferred to the tissue in the skin is deeper in the first operation mode and the depth at which heat is transferred to the tissue in the skin is shallower in the second operation mode, heat may be transferred to a deeper position in the skin to obtain an effect such as body fat reduction in the first operation mode, and heat may be transferred to a shallower position in the skin to obtain an effect such as skin beauty in the second operation mode.

As described above, the skin treatment device for thermotherapy according to an embodiment of the present invention may adjust a depth at which heat is transferred to tissues in the skin according to operation modes. The controller 500 can control the signal connector 420 to adjust the position of the common electrode among the plurality of electrodes in the electrode contact 300 so as to operate the first operation mode by making the distance between the common electrode and the operating electrode farther, or operate the second operation mode by making the distance between the common electrode and the operating electrode closer.

FIG. 3 shows a case where the fourth electrode 340 positioned at the edge of the electrode contact is used as the common electrode and the other electrodes are used as the operating electrodes to form the distance between the common electrode and one of the operating electrodes the farthest, in the first operating mode.

Even if the first electrode 310 located at the opposite edge is the common electrode instead of the fourth electrode 340 and the other electrodes are the operating electrodes, the distance between the common electrode and one of the operating electrodes may be the same as that shown in FIG. 3, and thus, substantially the same first operation mode may be operated.

Also, FIG. 4 shows a case where the third electrode 330 positioned in the middle of the electrode contact is used as the common electrode and the other electrodes are used as the operating electrodes to make the distance between the common electrode and one of the operating electrodes closer, in the second operating mode.

Even when the second electrode 320 is used as the common electrode instead of the third electrode 330 and the other electrodes are used as the operating electrodes, the distance between the common electrode and the operating electrode is the same as that shown in FIG. 4, and thus, the same second operation mode may be operated.

FIGS. 1 to 4 show the case where the electrode contact enabling in contact with the skin is configured such that multiple circular electrodes are concentrically formed at predetermined intervals.

Regardless of whether the electrodes provided in the electrode contact of the skin treatment device for thermotherapy according to the present invention are circular, straight, or any shape, if a plurality of electrodes are arranged at predetermined intervals to contact the skin, the electrode contact may operate according to substantially the same principle as shown in FIGS. 1 to 4.

In addition to the electrode contact having configuration of the pattern 1 type as shown in FIGS. 1 to 4, for example, various operation modes of the skin treatment device according to this invention may be implemented by using the electrode contact configuration of the pattern 2 type or the electrode contact configuration of the pattern 3 type.

In addition to *pattern 1 type of the electrode contact as shown in FIGS. 1 to 4, various operation modes of the skin treatment device according to the present invention may be operated even by using, e.g., *pattern 2 type of electrode contact (a pattern in which multiple electrodes in a straight line are arranged) or *pattern 3 type of electrode contact (a pattern in which multiple dot-shaped electrodes are arranged).

*pattern 1: [symbol] *pattern 2: [symbol] *pattern 3:

[symbol]

Meanwhile, in FIGS. 1 to 4, two operation modes of the first operation mode and the second operation mode are provided according to a depth at which heat is transferred to tissues in the skin using the electrode contact including four electrodes.

However, the present invention is not limited thereto, and three or more operating modes may be provided according to the depth of heat transfer to the tissue in the skin, and each operating mode may be operated by controlling the position of the common electrode for each operating mode.

As described above, the skin treatment device according to the present invention is a thermal therapy device in which radio-frequency electrical energy is transferred to electrodes in contact with the skin and heat is applied to tissue under the skin by electric fields formed by the electrodes.

The skin treatment device for thermotherapy according to the present invention has advantages in that it is possible to provide various operation modes such as an operation mode for skin beauty effects and an operation mode for reducing body fat by controlling a depth for generating heat under the skin by changing the positions of the bipolar electrode-pairs of the electrodes in contact with the skin,

What is claimed is:

1. A skin treatment device for thermotherapy comprising:
a handpiece;
a head provided on a distal end of the handpiece, wherein the head has an electrode contact including a plurality of electrodes in contact with a skin and transferring heat to tissues in the skin according to radio-frequency electric signal applied to each of the plurality of the electrodes;
a signal operator that generates and connects the radio-frequency electric signal to each of the electrodes of the electrode contact; and
a controller that controls the signal operator to make one of the electrodes into a common electrode and each of the other electrodes into an operating electrode so that the common electrode and each of the operating electrodes form a bipolar electrode-pair and the bipolar electrode-pair transfers heat by the radio-frequency electric signal to tissues in the skin, wherein the controller controls the signal operator to change a position of the common electrode.

2. The device according to claim 1, wherein the controller is configured to control the signal operator to adjust a depth at which heat is transferred by the radio-frequency in tissues in the skin by changing an electrode to which a signal for the common electrode is transmitted.

3. The device according to claim 1, wherein the signal operator comprises:
a signal generator that generates an electric signal for the common electrode and an electric signal for each of the operating electrodes; and
a signal connector that connects an electrical signal for the common electrode to an electrode specified by the controller among the plurality of the electrodes of the electrode contact and connects electrical signals for each of the plurality of operating electrodes to the other electrodes other than the specified electrode, respectively, so that the common electrode and each of the operating electrodes form a bipolar electrode-pair.

4. The device according to claim 1, wherein the controller is configured to provide a plurality of operation modes according to a depth at which heat is transferred by the radio-frequency in tissues in the skin, and control the signal operator to allow an electric signal for the common electrode to be connected to an electrode selected from the plurality of the electrodes of the electrode contact according to an operation mode selected from the plurality of the operation modes.

5. The device according to claim 4, wherein the controller is configured to control the signal operator to change the position of the common electrode so that the distance between the common electrode and at least one of the operating electrodes forming the bipolar electrode-pair is farther as the depth of heat transferred to the tissue in the skin is deeper.

6. The device according to claim 1, wherein the controller is configured to control the depth of heat generated by the electrode contact in the tissue in the skin by adjusting the distance between the common electrode and each of the operating electrodes forming the bipolar electrode-pair.

7. The device according to claim 1, wherein the signal operator includes a plurality of channels so that each of the plurality of the channels is connected to each of the plurality of electrodes of the electrode contact.

8. The device according to claim 7, wherein the signal operator is configured to transmit the radio-frequency electrical signal to each electrode through each channel according to the control of the controller.

9. The device according to claim 7, wherein the signal operator is configured to transmit an electric signal for the common electrode through a channel selected to make an electrode selected by the controller into the common electrode.

10. The device according to claim 7, wherein the signal operator is configured to allow one of the plurality of electrodes of the electrode contact to be the common electrode through one of the channels, and allow each of the other electrodes of the electrode contact to be the operating electrode through each of the other channels, so that the common electrode and each of the operating electrodes form a bipolar electrode-pair.

11. The device according to claim 1, wherein the signal operator is configured to apply AC current the common electrode and each operating electrode as bipolar electrode-pairs so that a direction of electric fields of the bipolar electrode-pairs are consecutively changed and generate deep heat in the tissue in the skin.

12. The device according to claim 1, wherein the electrode contact of the head includes a plurality of circular electrodes as the plurality of the electrodes and is configured such that the plurality of circular electrodes form a concentric circle at predetermined intervals,
wherein the controller is configured to control the signal operator to make a circular electrode selected from the plurality of the circular electrodes into the common electrode and make each of the other circular electrodes into the operating electrode so that the common electrode and each of the operating electrodes form a bipolar electrode-pair, and
wherein the controller is configured to control the depth at which heat generated by the bipolar electrode-pair is transferred in the tissue in the skin by adjusting a distance between the common electrode and the operating electrode.
